(19) United States
(12) Patent Application Publication
(10) Pub. No.: US 2006/0161211 A1
(43) Pub. Date: Jul. 20, 2006

(54) Implantable accelerometer-based cardiac wall position detector

(76) Inventors: Todd Thompson, San Jose, CA (US); George Savage, Portola Valley, CA (US); Olivier Colliou, Los Gatos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/324,196

(22) Filed: Dec. 29, 2005

Related U.S. Application Data
(60) Provisional application No. 60/640,450, filed on Dec. 31, 2004.

(51) Int. Cl. A61N 1/368

(52) U.S. Cl. 607/19; 607/17

(57) ABSTRACT

Methods for evaluating motion of a cardiac tissue location, e.g., heart wall, are provided. In the subject methods, timing of a signal obtain from a accelerometer stably associated with the tissue location of interest is employed to evaluate movement of the cardiac tissue location. Also provided are systems, devices and related compositions for practicing the subject methods. The subject methods and devices find use in a variety of different applications, including cardiac resynchronization therapy.

### CROSS-REFERENCE TO RELATED APPLICATIONS

This application claims priority under 35 U.S.C. §119(e) to U.S. Provisional Application Ser. No. 60/640,450 filed on Dec. 31, 2004, the disclosure of which is herein incorporated by reference.

### BACKGROUND OF THE INVENTION

The present Invention relates generally to medical methods, apparatus and systems. More specifically, the invention relates to methods, apparatus and systems for optimizing cardiac resynchronization intervention, arrhythmia management, ischemia ejection, coronary artery disease management, and heart failure management.

Cardiac resynchronization therapy is an important new medical intervention for patients suffering from congestive heart failure. In congestive heart failure, symptoms develop due to the inability of the heart to function sufficiently well as a mechanical pump to supply the body's physiological needs. Congestive heart failure is characterized by gradual decline in cardiac function punctuated by severe exacerbations leading eventually to death. It is estimated that over five million patients in the United States suffer from this malady.

The aim of resynchronization pacing is to induce the interventricular septum and the left ventricular free wall to contract at approximately the same time. Resynchronization therapy seeks to provide a contraction time sequence which will most effectively produce maximal cardiac output with minimal total energy expenditure by the heart. The optimal timing is calculated by reference to hemodynamic parameters such as dP/dt, the first derivative of the pressure waveform in the left ventricle. The dP/dt parameter is a well-documented proxy for left ventricular contractility.

In current practice, external ultrasound measurements are used to calculate the dP/dt. Such external ultrasound is used to observe wall motion directly. Most commonly, the ultrasound operator uses the ultrasound system in a tissue Doppler mode, a feature known as tissue Doppler imaging or TDI, to evaluate the time course of displacement of the septum relative to the left ventricle free wall. The current view of clinicians is that ultrasonographic evaluation using TDI or a similar approach may become an important part of qualifying patients for CRT therapy.

As currently delivered, CRT therapy is effective in about half to two-thirds of patients implanted with a resynchronization device. In approximately one third of these patients, this therapy provides a two-class improvement in patient symptoms as measured by the New York Heart Association fair level scale. In about one third of these patients, a one-class improvement in cardiovascular symptoms is accomplished. In the remaining third of patients, there is no improvement or, in a small minority, a deterioration in cardiac performance. This group of patients are referred to as non-responders. It is possible that the one-class New York Heart Association responders are actually marginal or partial responders to the therapy, given the dramatic results seen in a minority.

The synchronization therapy, in order to be optimal, targets the cardiac wall segment point of maximal delay, and advances the timing to synchronize contraction with an earlier contracting region of the heart, typically the septum. However, the current placement technique for CRT devices is usually empiric. A physician will cannulate a vein that appears to be in the region described by the literature as most effective. The device is then positioned, stimulation is carried out, and the lack of extra cardiac stimulation, such as diaphragmatic pacing, is confirmed. With the currently available techniques, rarely is there time or means to attempt to optimize cardiac performance.

When attempted today, CRT optimization must be preformed by laborious manual method of an ultrasonographer evaluating cardiac wall motion at different lead positions and different interventricular delay (IVD) settings. The IVD is the ability of pacemakers to be set up with different timing on the pacing pulse that goes to the right ventricle versus the left ventricle. In addition, all pacemakers have the ability to vary the atrio-ventricular delay, that is the delay between stimulation of the atria and the ventricle or ventricles themselves. These settings can be important in addition to the location of the left ventricular stimulating electrode is itself in resynchronizing the patient.

Some research efforts to assess cardiac motion through internal sensors, e.g. accelerometers, have been made. Pacesetter researchers have described use of epicardial accelerometry as an arrhythmia detection system. Kroll et. al. teach a positional accelerometer for rate control (U.S. Pat. No. 6,625,493, issued Sep. 23, 2003). Mouchawar et. al. disclose cardiac wall motion detection using an accelerometer for detection of arrhythmias (U.S. Pat. No. 6,002,963, issued Dec. 14, 1999). Park et. al. teach the use of an accelerometer for rate adaptive pacing (U.S. Pat. No. 5,991,661, issued Nov. 23, 1999). Nilsson describes an in-can accelerometer to provide rate control (U.S. Pat. No. 6,044,299, issued Mar. 28, 2000).

Other research groups have explored the use of accelerometry in cardiac applications. Carlson et. al. teach use of an in-can accelerometer which derives pulse pressure for pacing (CRT) optimization using signals from an accelerometer and an ECG (U.S. Pat. No. 6,366,811, issued Apr. 2, 2002). Salo et. al. teach the use of an accelerometer with signal processing circuitry to measure total acoustic notes to optimized CRT (U.S. Pat. No. 6,058,329, issued May 2, 2000). Cunningham teaches use of an accelerometer in the ventricle on a lead to monitor cardiac contractility (U.S. Pat. No. 6,077,136, issued Jun. 20, 2000).

Current of accelerometry in cardiac applications include implantable accelerometers for determining patient activity levels. With these devices, the pacemaker paced rate can adjust itself to allow for exercise and greater physical activity on the part of a pacemaker-dependent patient.

Recently, Overall et. al. have described the concept of using apical accelerometry and other sensors to detect heart ischemia by detecting abnormalities in motion (WO 2004/066825 A2, published Aug. 12, 2004). Yu et. al. have described the use of one axis accelerometers alone to note difference in the synchronicity of ventricular wall location contractions (US 2003/0105496 A1, published Jun. 5, 2003).

Some researchers have reported the use of position sensors deployed along different aspects of the heart with the intent of delivering relative position with the aim of describing the extent of myocardial contraction and effectively duplicating a portion of the information provided typically through ultrasonographic means in the clinic today. Such parameters include ejection fraction, stroke volume, cardiac output, and synchronization index. These systems use a fixed frame of reference using ultrasonographic, magnetic or RF fields in orthogonal planes to generate a signal which can localize a catheter or catheters within the heart.

There is currently no useful clinically available means of determining optimal CRT settings on a substantially automatic or a real-time, machine readable basis. It would be an important advancement in cardiology to have an implantable means of monitoring the mechanical performance of the heart in real time, an immediate application being in setting the functions of cardiac resynchronization therapy pacemakers with further application to the pharmacologic management of heart failure patients; arrhythmia detection and ischemia detection.

### SUMMARY OF THE INVENTION

The inventive implantable accelerometer-based cardiac wall position detector system allows, for the first time, the use of accelerometers for purposes of tracking cardiac wall motion. The present inventive implantable wall position detector provides a critical new tool in the physician's armamentarium which provides accurate, real time monitoring of the mechanical performance of the heart.

An immediate and particularly important application for this technological breakthrough is to provide the optimum function settings for cardiac resynchronization therapy pacemakers. Employing the present inventive implantable wall position detector device and method, optimal resynchronization can be accomplished on a manual basis employing the more basic embodiments of the present invention. Additionally, in more complex embodiments, the present inventive implantable wall position detector provides optimal resynchronization data into the algorithm for automatic setting of cardiac resynchronization therapy pacemakers.

The immediate goal of the present invention is to obtain hemodynamic parameter data. The current use of Doppler in cardiac imaging is accomplished by an externally applied ultrasound beam which is used to create an image. This image can, on further processing, be employed to derive important hemodynamic parameters. These parameters can provide some indication of cardiac wall synchronicity important to optimizing clinical performance of a biventricular or cardiac resynchronization pacemaker.

The present inventive implantable wall position detector system allows, for the first time, the availability of effective resynchronization intervention to the significant population of non-responding patents who currently do not benefit from this life-saving therapy.

The present innovation of employing one or more accelerometers in the inventive implantable wall position detector to describe wall motion for purposes of resynchronization of a cardiac resynchronization therapy device is unique. Therefore, additional applications of this critical tool to various clinical challenges will be appreciated by the clinicians, as will many unique approaches enabled by this new tool.

For instance, the data provided by the inventive implantable wall position detector can be provided directly to the physician's office for monitoring patent progress, and providing modification of pharmaceutical intervention without requiring patient travel. This would be particularly advantageous for patients in remote areas, or locations without access to skilled ultrasound technicians.

Additionally, using the implantable wall position detector, physicians will be able to monitor patients during normal daily activities. This advancement will encourage heart failure patients to resume health promoting increase in physical exertion. In some cases, such patients would, for the first time, be able to undertake a program of increasingly active exercise that would increase the quality of their lives and improve their overall clinical improvement.

In the context of the present application, the term “accelerometer” includes within this meaning the use of existing electrodes or other cardiac elements which can serve in an accelerometer capacity in the context of the overall inventive system. Thus, current available motion sensors can serve as “accelerometers” within the inventive system even if they were not initially designed or implanted to serve in that capacity.

By employing a variety of accelerometers in the present implantable wall position detector system, each at a different position within or externally to the heart, multiple positions and velocity can be calculated from differing reference frames. This embodiment of the present invention creates an accelerometry based wall motion profile providing an enormous amount of clinically relevant velocity and positional information in real-time.

As a major advancement over currently available clinical ultrasound methods, these data provided by the inventive implantable wall position detector system is inherently machine-useable as the positioning velocity data are numeric. Currently available data is limited to an image requiring human interpretation with all the inconsistencies inherent in individual interpretation.

The present inventive implantable wall position detector can be implemented in the practical deployment of multiple sensors to describe in further detail wall motion on a segmental basis. This novel platform multiplexed endocardial lead system is an innovation of some of the current inventors, and is described below with reference to co-pending and published applications.

### SUMMARY OF THE DRAWINGS

FIG. 1 provides a diagram of an embodiment of the inventive implantable wall position detector.

FIG. 2 provides a diagram of the view in FIG. 1, showing additionally the cardiac motion.

FIG. 3A-3B. FIG. 3A provides a view of the inventive implantable wall position detector with pacing leads. FIG. 3b provides a three dimensional cutaway view of placement of the inventive implantable wall position detector system in the left ventricle.

FIG. 4A-4B. FIG. 4A shows accelerometer leads in a cross section of the heart, and FIG. 4B show a close up of their placement in the coronary septum.

FIG. 5A-5C. FIG. 5A is a wireless battery less accelerometer sensor, FIG. 5B a wireless pressure sensor of a similar design, and FIG. 5C shows a device designed for placement in the cardiac vein.

FIG. 6 shows placement of wireless sensors in various locations in the heart.

FIGS. 7A-7D provide views of the inventive device as a shaped set lead with multiple displacement sensors and/or electrodes.

FIGS. 8A-8F provide cross-sectional views of various accelerometer lead embodiment of the inventive cardiac timing device.

### DETAILED DESCRIPTION OF THE INVENTION

The inventive implantable wall position detector system uses relative acceleration of cardiac features to determine the relative position of various cardiac structures.

The implantable wall position detector method of the present invention is in some ways similar to ultrasound used in the clinical environment. However, by employing a variety of accelerometers, multiple relative positions and velocity can be calculated from differing reference frames. Thus, an accelerometer map of the wall position is created. This unique data providing, for the first time, clinically relevant velocity and positional information in real time. This data is inherently machine-useable as the positioning velocity data are numeric rather than an image requiring human interpretation with all the inconsistencies inherent in individual interpretation.

In biventricular or cardiac resynchronization pacing applications of the present implantable wall position detector invention, relative wall motion determined by the present invention allows optimization of synchrony. In this embodiment of the present invention, coordinated speeds and directions provided from the accelerometers indicates maximal synchrony between the cardiac features in which they are position. For instance, an accelerometer positioned against the right ventricular septum as compared with another accelerometer positioned in an optimal cardiac vein located in the epicardial aspect of the left ventricular free wall will provide the necessary information.

Method of Wall Motion Determination

In the present inventive implantable wall position detector, an inverse direction but coordinated acceleration indicates maximal relative motion inward between two cardiac features. To optimize the use of the data obtained by the present invention, an additional embodiment can include correlating this data with data from a pressure sensor. This pressure can be located in either the right or left ventricle or nearby in another area such as the aorta. Thus, this embodiment of the inventive implantable wall position detector can include coordinating data provided by a pressure sensor indicating systolic performance with the wall motion data providing improved hemodynamic performance for congestive heart failure patients.

By adding a pulsing feature and time of flight information to the accelerometer component of the inventive implantable wall position detector, direct positional information is added to the mix of information. In this manner, synchrony is assessed between various parameters such as a dP/dt, the first derivative of the pressure curve correlated to maximal relative velocity during systole towards the center of the ventricle. Also provided is the actual maximum position of displacement on a net basis of the monitored wall segments towards the center.

Synchrony and Other Applications

The optimal mix of the various parameters provided by the present implantable wall position detector, which will be readily determinable by the practioner, allows for calibration of the optimal stimulation location or locations in the setting of CRT. The inventive implantable wall position detector also provides for optimal timing of the sequence of atrial ventricular and ventricle to ventricular delay timings. In addition, multiple resynchronization electrodes are stimulated in a pattern and in manners previously unavailable to the clinician and as yet undescribed. This is due to the current unavailability of the inventive optimization and feedback system made possible by the inventive implantable wall position detector.

Additional applications for the inventive implantable wall position detector include applications as an ischemia detector. It is well understood that, before biochemical or electrical markers of cardiac ischemia present themselves, wall motion is first affected. This displays with the ischemic region showing increased stiffness and decreased contraction. These types of changes can be readily detected by the system currently invented.

The inventive implantable wall position detector system can serve as an arrhythmia detector. Currently implantable defibrillator systems are challenged by differentiating between a variety of benign and malignant arrhythmias relying as they do primarily on electrical means of discrimination. Mechanical means, such as the wall motion positional detector, of the subject invention marks a significant advantage in detecting arrhythmias.

In the management of congestive heart failure patients, the inventive implantable wall position detector has clinical capacity well beyond the needs of optimizing either on a one-off basis or dynamically as part of a closed loop feedback system for biventricular pacing. The inventive implantable wall position detector can be effectively employed by specialists, such as congestive heart failure cardiologists, to address a patient's medication, diet and exercise regimen in response to real time physiologic data such as ejection fraction, stroke volume, and cardiac output determined by the inventive system.

The totally implantable wall position detector system embodiment of the present invention includes motion detectors and other structures which can provide accelerometry information. This system can be further modified in another embodiment of the present invention if relative positional data from such a system prove inadequate for certain cardiovascular applications.

In additional embodiments of the present invention, computation ornaments are added to the system even on an implantable basis for full time analysis or via download or real time interrogation on an external basis in order to compute the parameters of interest at any given time.

An integrated, multi-axis accelerometer implantable wall position detector with positional orientation is presented for the first time herein in the context of an implantable cardiac device. The current inventive implantable wall position detector offers both a solidstate and constructible, reliable means of optimizing biventricular pacing both in terms of location and timing. This allows prompt detection of reversible and irreversible ischemia, especially so-called “silent ischemia”.

The inventive implantable wall position detector also allows a determination of important hemodynamic parameters on a permanent implantable basis. Such hemodynamic parameters can include such components as stroke volume, ejection fraction, cardiac output and others, as well arrhythmia detection and classification via reliable mechanical means.

Using the devices and methods of the present inventive implantable wall position detector system, the timing and displacement of contraction of the monitored sections of the heart can be compared to one another, phase and amplitude differences evaluated, and means manually or automatically taken to move contraction of wall segments into synchronization with one another. In this way, the maximum contraction occurs at essentially the same time or the time most efficient from the standpoint of producing the greatest hemodynamic output for the least amount of effort.

Physical Description of Accelerometer—Attachment, Dimensions

When the accelerometers of the present implantable wall position detector invention are designed for implantation in the cardiac vein using catheter methods, the sensor is about 0.25 mm in width, preferably about 0.25 to 1.5 mm wide and most preferably about 0.25 to 1.0 mm wide. A preferred dimension for the inventive implantable wall position detector design embodiment for implantation into a cardiac vein will typically be smaller, with the width dimension typically less than about 1.25 mm.

The inventive implantable wall position detector accelerometer is typically rectangular in shape, although it can take other forms as well. In inventive embodiments where physical orientation is preferred, the sensor can be manufactured in a shape that would allow orientation using an external system such as an x-ray. For example, a trapezoid shaped sensor would allow visualization of specific orientation for an axis of the sensor. This ability to provide orientation finds greatest use in single axis embodiment of the present invention. In that case, the long dimension of the inventive accelerometer is about 0.25 to 10.0 mm, preferably about 0.5 to 5.0 mm, and most preferably about 1.0 mm to 5.0 mm.

In the present inventive implantable wall position detector, an embodiment is provide combining multi-axis with single axis accelerometers. This embodiment of the present invention provides important clinical data. For example, a three axis accelerometer is located in or near the “can”, advantageously as space for positioning is readily available. The information from the accelerometer at the can is then subtracted from the signal measured at the accelerometers positioned in intra-cardiac locations to remove the motion not directly related to cardiac motion. This embodiment of the present inventive implantable wall position detector is most effective when sensors are oriented when placed. A single axis measurement on the wall of the heart is sufficient to provide the necessary information.

An additional embodiment of the present inventive implantable wall position detector employs a two axis accelerometer which is positioned on the heart wall. One axis is positioned to measure the inward and outward contraction motion of the heart. The second axis is positioned to measure the apical to basal motion of the heart. The challenge, in some cases, to this inventive embodiment is that the cardiac vein runs both laterally and towards the apex. However, a three axis accelerometer can be deployed and the axis of interest could be used only. A visual indication of orientation allows the choice of axis of interest. The latter is a more complex approach, and may not be suitable for standard procedures.

The accelerometers of the present inventive implantable wall position detector can be attached to various cardiac features by active fixation or passive fixation. Active fixation relies on various devices and methods to hold the accelerometer implantable wall position detector device in a preferred position. In some cases, active fixation is accomplished indirectly by fixing the lead to which the accelerometer is attached to the cardiac feature of interest. This can be accomplished by the use of screws and other devices, some which are intrinsic design features of the lead or the accelerometer. Current active fixation methods include helical screws, clips, staples, and other such devices.

The passive fixation of the inventive implantable wall position detector accelerometers provides methods which do not require the disruption of tissue. Tines are one method which can be used to capture leads in the right ventricle. The tines become tangled in the trabeculae carneae of the ventricle and allow the lead to become encapsulated by tissue over time. Other passive fixation approaches appropriate to the present invention includes the use of pre-formed leads in the cardiac venous system. A lead is pre-shaped into an S or helical shape. The stylet used to position the lead straightens it for placement. When removed, the lead the expands against the surrounding vessel, gently anchoring it in place.

Controlled positioning of the inventive implantable wall position detector accelerometers is typically most useful if 1 or 2 axis sensors are deployed. For example, if a single axis sensor is used, it is desirable in the present invention to have the sensor positioned in alignment with the axis of the most motion. The axis of greatest motion is typically aliened with the inward and outward contraction motion of the heart. Advantageously, this axis is the easiest on which to orient the inventive accelerometer.

Various means can be provided to insure proper positioning of the inventive implantable wall position detector accelerometers. For example, the lead can be twisted and otherwise maneuvered until the highest motion readings are displayed by the user interface. As an additional example, a simple thermometer style indicator can be shown to highlight the position of highest motion sensing.

The accelerometer based implantable wall position detector system of the present invention is tolerant of remodeling changes which may modify heart dimensions relative to the time of placement. This is because absolute cardiac feature position is not a major issue with the accelerometer data produced with the inventive system. Rather, relative motion and displacement are measured.

It is unlikely that heart remodeling would reposition the inventive sensor out of alignment with the axis of highest motion. Remodeling, however, can change the amplitude of motion. In that case, the user interface will be required to capture that information and display it in a useful way to the practitioner.

In certain embodiments of the present inventive implantable wall position detector, calibration of the sensor can be accomplished at the level of the can by attaching a sensor to the patient's skin adjacent to the can and comparing the data. This insures that the data which is subtracted for the calculation is accurate

Devices Positioned in the Heart

In one embodiment of the present invention, the critical resynchronization data is obtained by means of localizing endocardial elements along the right ventricular septum and an aspect of the left ventricle. This can be accomplished either by the endocardial approach through a cardiac vein or through an epicardial approach analogous to placement of an epicardial left ventricular stimulation electrode. The inventive device in this case is configured to describe the relative position of the different wall segments relative to one another.

A preferred embodiment of this approach involves the placement of one or more accelerometers along a lead located in close association with the right ventricular septum. In addition, a lead may be located in a cardiac vein located on the left ventricular surface. An alternative to this approach includes a lead using an accelerometer placed in the antero-septal vein that roughly tracks the interventricular septum and another accelerometer further laterally or posteriorly along the left ventricular surface.

In another aspect of the present invention, additional accelerometers are place along the aspect of the right ventricular free wall. This provides an understanding of interventricular disynchrony, rather than intraventricular disynchrony within the left ventricle itself. These data are particularly useful in cases of both right ventricular heart failure and right-sided heart failure.

Use of the present inventive accelerometer based implantable wall position detector system can be usefully coordinated with other positional sensors. These additional positional sensors include electromagnetic methods such as tuned circuits, Hall effect sensors, time-of-flight sensors, Doppler systems, and ultrasonographic methods among others. The coordinated use of these various positional sensors can be accomplished using a number of different approaches. For example, a multi-electrode left ventricular pacing lead can automatically select the optimal electrode and optimal timing sequence within a predetermined range selected by the physician using important hemodynamic parameters indicative of resynchronization.

Such additional sensors have been described in various applications by some of the present inventors. These applications as also describe multiplexing systems previously developed by some of the present inventors with which the present invention is very usefully employed.

This prior work by some of the present inventors describes the use of dimension sensors for resynchronization. This system is described in part in currently pending patent applications U.S. patent application Ser. No. 10/764,429 entitled “Method and Apparatus for Enhancing Cardiac Pacing”, U.S. patent application Ser. No. 10/764,127 entitled “Methods and Systems for Measuring Cardiac Parameters”, and U.S. patent application Ser. No. 10/764,125 entitled “Method and System for Remote Hemodynamic Monitoring” all filed Jan. 23, 2004, U.S. patent application Ser. No. 10/734,490 entitled “Method and System for Monitoring and Treating Hemodynamic Parameters” filed Dec. 11, 2003, and U.S. Provisional Patent Application entitled “Methods and Systems for Programming and Controlling a Cardiac Pacing Device” filed Dec. 23, 2004. These applications are herein incorporated into the present application by reference in their entirety.

Additional positional sensors for use in the present implantable wall position detector system can include but are not limited to, electromagnetic methods such as tuned circuits, Hall effect sensors, time of flight sensors, Doppler, continuous field tomography, and ultrasonographic methods. The present inventive implantable wall position detector system can incorporate a number of other sensing capabilities which augment, in many cases synergistically, the use of strain gauges in the present inventions.

Some of the present inventors have developed Doppler, pressure sensors, additional wall motion, and other cardiac parameter sensing devices. Some of these are embodied in currently filed applications; “Methods And Apparatus For Tissue Activation And Monitoring” PCT application filed Sep. 1, 2005, attorney docket No. 021309-001340PC, “Continuous Field Tomography” PCT application filed Oct. 6, 2005, attorney docket No. 021308-001610PC, “Cardiac Motion Characterization by Strain Measurement” U.S. Ser. No. 05/39535, “Implantable Pressure Sensors” PCT application filed Dec. 10, 2004, “Pressure Sensors Having Stable Gauge Transducers” U.S. Ser. No. 11/025,876, “Pressure Sensor Circuits” U.S. Ser. No. 11/025,366, “Pressure Sensors Having Transducers Positioned to Provide for Low Drift” U.S. Ser. No. 11/025,879, “Pressure Sensors Having Neutral Plane Positioned Transducers” U.S. Ser. No. 11/025,795, “Implantable Pressure Sensors” U.S. Ser. No. 11/025,657, “Pressure Sensors Having Spacer Mounted Transducers”, U.S. Ser. No. 11/025,793 all filed Dec. 28, 2004, “IMPLANTABLE HERMETICALLY SEALED STRUCTURES” PCT application filed Dec. 22, 2005, attorney docket No. PRTS-011WO, “Implantable Addressable Segmented Electrodes” PCT application filed Dec. 22, 2005, attorney docket No. PRTS-017WO. These applications are incorporated in their entirety by reference herein.

Other derived hemodynamic parameters similar to those obtained in ultrasound will be recognized by the artisan. In an additional embodiment of the present invention, additional sensors deployed along other areas of the heart in multiple planes provide data that will provide a complete characterization of the function of the ventricle or ventricles. This wealth of real time information can be provided continuously to the clinician on a permanent implantable basis. This ongoing position data can also be provided to the pacing system controller directly. This allows automated optimization of pacing timing and to which electrodes pacing will prove most clinically beneficial.

As described above, an important application of the present inventive implantable wall position detector is in cardiac resynchronization, or CRT, also termed biventricular pacing. The CRT technique has proven effective in relieving symptoms, improving quality of life, and prolonging life in selected congestive heart failure patients. Currently, CRT therapy is medically indicated in approximately 15% of the CRT population. This population is defined as patients suffering from moderate to severe heart failure, New York Heart Association Class III/IV, a prolonged QRS interval of greater than 150 ms, an enlarged left ventricle, and a left ventricular endostolic dimension of 50 mm or greater.

Cardiac resynchronization therapy attempts to remedy the delayed left ventricular mechanics of heart failure patients. In a desynchronized heart, the interventricular septum will often contract ahead of portions of the free wall of the left ventricle. In such a situation, where the time course of ventricular contraction is prolonged, the aggregate amount of work performed by the left ventricle against the intraventricular pressure is substantial. However, the actual work delivered on the body in the form of stroke volume and effective cardiac output is lower than would otherwise be expected.

Biventricular pacing therapy typically introduces pacing leads in the right atrium, right ventricle, and the left ventricle. 90% of such patients have the left ventricular lead placed via the right side of the heart. The endovascular method accesses the coronary sinus and thence along the coronary sinus to a branch of the cardiac vein. The preferential position is along the lateral or posterior-lateral aspect of the left ventricular free wall.

In approximately 10-15% of patients, adequate cardiac venous access for introduction of pacing leads is not available. In these cases, left ventricular stimulation must be performed via placement of an epicardial lead, usually via a minimally invasive surgical procedure.

CRT is the first device-based therapy for CHF which has been demonstrated to improve patient quality of life. It has been characterized by extremely rapid growth in number of units implanted since its introduction.

There are disadvantages to CRT therapy as currently administered. It is very expensive, with the average price for CRT systems in the field being approximately $32,000, and in some cases higher. This price provides for very capable systems which incorporate anti-tachycardia and ventricular fibrillation therapies such as anti-tachycardia pacing and defibrillation. This capability may save the patient in the event of a lethal arrhythmia in addition to the beneficial resynchronization pacing provided.

As currently practiced, external ultrasound measurements are used to calculate the dP/dt. Such external ultrasound is used by the clinician to directly observe wall motion. Each ultrasound operator uses the ultrasound system in a tissue Doppler mode, a feature known as tissue Doppler imaging or TDI, to evaluate the time course of displacement of the septum relative to the left ventricle. In this case, a desynchronized heart will develop a dyskinetic motion of part of the ventricle relative to another. As a result, as the septum contracts, the clinician can observe that the left ventricle free wall ventricle, far from contracting, actually move outwards in the direction opposite appropriate function. The mechanical effect of this is to move a portion of the left ventricular blood from one side to the other rather than effectively squeezing it outward into the aorta as required for optimized function.

The present inventive implantable wall position detector represents an important advancement in resynchronization intervention by extending the population of patients benefiting from CRT therapy well beyond the current group of CHF responders to this therapy. There is a growing realization in cardiac medicine that QRS interval may be a non-optimal criteria to rely upon in resynchronization patients. A prolonged QRS represents slow electrical depolarization of the heart, electrical disynchrony, which does not always correlate to mechanical disynchrony. There is growing appreciation that a large number of patients have mechanical disynchrony in the setting of a normal QRS interval. These patients display a normal depolarization event followed by a variable response to that depolarization event in terms of the timing sequence of ventricular contraction.

A preferred embodiment of the present inventive implantable wall position detector is configured as an implantable system with either a can, hermetically sealed can with a battery and processing gear, or a coil designed for subcutaneous placement. With this inventive configuration, power and data can be transmitted through the skin to the device. Two leads extend from the inventive device. One of these leads is placed in the right ventricle in close association with the interventricular septum. The second lead is positioned to access the coronary sinus by being placed along another aspect of the left ventricle through a cardiac vein. Alternatively, the leads can be positioned in a manner analogous to the cardiac resynchronization therapy process. For instance, a left ventricular lead can be placed epicardially if suitable cardiac veins are not available for cannulation.

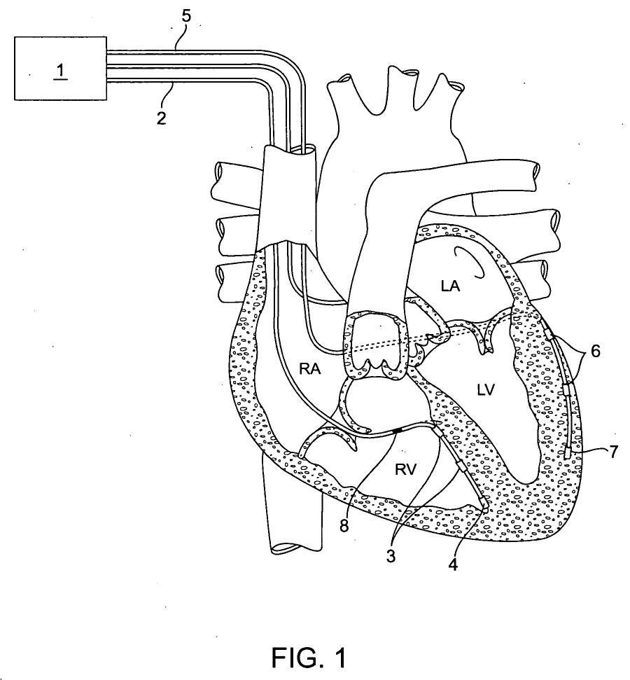

FIG. 1 provides a diagrammatic view of a preferred embodiment of the inventive implantable wall position detector system. Communication means 1 provides the extracardiac communication and calculation means for the overall system. Communication means 1 can take the form of various embodiments including an implantable device complete with power supply, drive electronics and processing power on board. In more complex configurations, communication means 1 may provide a means for communicating data and power from a completely external or extracorporeal location.

Right ventricular lead 2 emerges from communication device in communication means 1, and travels from the preferentially subcutaneous location of communication means 1 via the subclavian venous access through the superior vena cava through the right atrium and then through the tricuspid valve to a position along the right ventricle. This location is preferentially located along its distal portion in close association with the intraventricular septum terminating distally with fixation in the right ventricular apex.

Particular to distal aspect of right ventricular lead 2 are right ventricular accelerometers 3 and 4. In other embodiments of the present invention, an additional number or smaller number of accelerometers may be employed.

Additionally emerging at the proximal aspect of communication means 1 is left ventricular lead 5. Left ventricular lead 5 starts by following the same route as right ventricular lead 2 via subclavian vein through the superior vena cava into the right atrium. At this point, left ventricular lead 5 is placed via the coronary sinus around the posterior aspect of the heart and thence into cardiac vein draining into said sinus.

FIG. 1 further depicts left ventricular lead 5 in a position likely to be advantageous for biventricular pacing located along the lateral aspect of the left ventricle. Left ventricular accelerometers 6 and 7 are shown in this drawing analogous to accelerometers 3 and 4 which are previously described.

Right ventricular lead 2 may optionally be provided with pressure sensor 8 which is located in the right ventricle. Pressure sensor 8 provides a pressure signal which can also simultaneously be obtained with wall motion data. It is notable that adding active devices to said lead such as pressure sensor 8 is facilitated through use of a multiplexing system, which has been previously disclosed and may or may not be used in this case.

Principle of operation of the inventive implantable accelerometer based implantable wall position detector system is that a communication means 1 will communicate with each of the accelerometers. Each accelerometer is provided a signature communication. These communications can be delivered with a clock used to determine the onset of the signal. Then detection is timed for the various accelerometer data. Simultaneously, in the current example of the communication being received at left ventricular accelerometers 6 and 7 would be routed back to communication means 1.

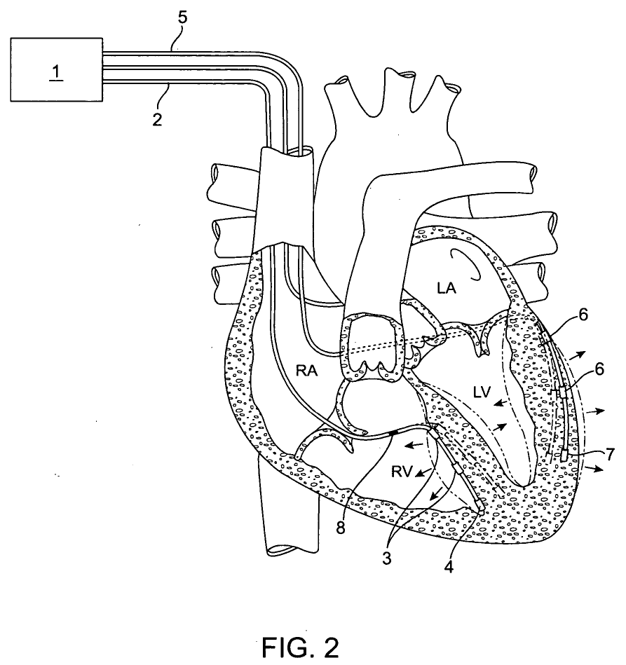

FIG. 2 depicts the roles of the inventive accelerometers when heart is in motion. With a lead such as right ventricular lead 2 and left ventricular lead 5 is provided in close association with the wall of the heart. As the wall of the heart moves via 3D cardiac cycle, so do the catheters in a proportionate amount. As these catheters move towards and away from one another, the range and velocity information derived from the aforementioned method shifts over the course of the cardiac cycle. This shift is indicative of their movement and timing of said movement.

The position data together with an optional pressure signal or signals is used to optimize cardiac resynchronization therapy where the goal is to maximize the contractility of the left ventricle. This is obtained by encouraging effectively simultaneous contraction of the bulk of the muscle of the left ventricle. In many congestive heart failure patients, such contractility is impaired with dyskinetic contraction often occurring typically septal contraction against a relaxed left ventricle followed by ventricular contraction against a relaxing septum. The result is inefficiency with regards to blood being moved around the ventricle rather than constrictively expelled from the ventricle as in a normal human dynamic case.

Current systems provide biventricular pacing on an empiric basis only where optimization is attempted. It is generally a time-consuming process relying on external cardiac ultrasound. A physician attempts to visualize wall motion. With the aid of tools in the ultrasound machine, the physician calculates a synchronicity index based on the wall motion just described. The current system would provide such data in real time and in a numeric format useable by both the implanting physician and by an automated pacing system such as a CRT device.

The inventive implantable wall position detector system can detect abnormalities in wall motion associated with ischemia or arrhythmia and other cardiac abnormalities including progression of underlying disease states such as congestive heart failure. If connected to an external or fully implanted real time monitor, such abnormalities will trigger an alarm. This alarm alerts the patient and or their physician of the advent of these abnormalities.

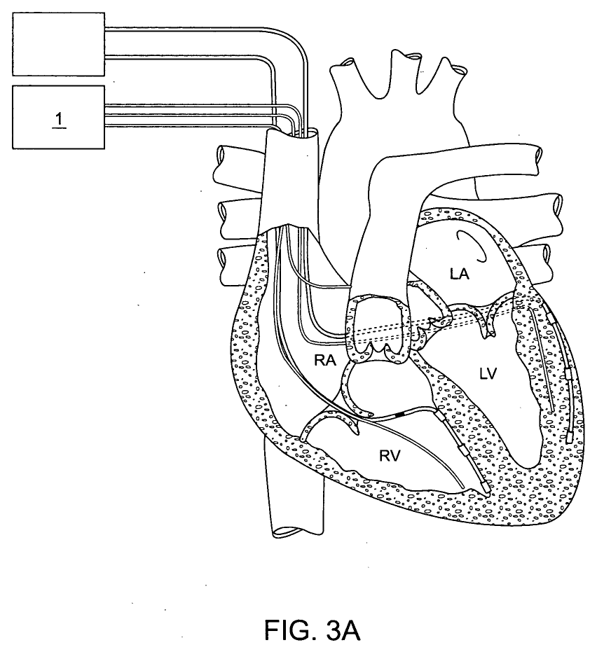

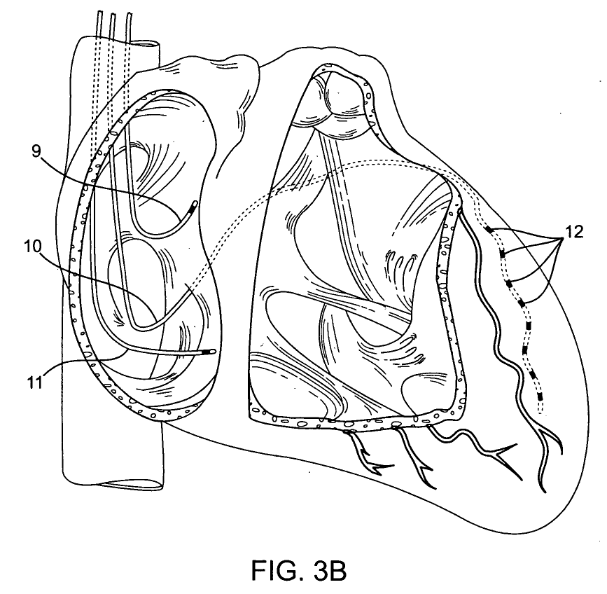

FIG. 3A shows an additional view of the heart, including pacing leads. In this case, three leads are depicted which would be the typical state in a biventricular pacing system in which the current invention could be integrated in another preferred embodiment.

FIG. 3B depicts graphically in right atrial lead 9 which is a right atrial pacing lead. A left ventricular lead 10 is depicted entering the coronary sinus. The dash lines indicate passage through the coronary sinus and thence along the interior of a cardiac vein along the left ventricular surface. Right ventricular lead 11, while not shown the current view, is preferential positioned intimately along the intraventricular septum by means of the various accelerometers 12 along the left ventricular lead 10. This information could be taken relative one to another to give a sense for local left ventricular shortening as well as relative to the accelerometers located in the right atrium and right ventricle.

In practice, each accelerometer may be provided with a microprocessing chip. When appropriate for the can to receive this information, the data is distinguished as being received from a particular accelerometer by a unique address. When multiple accelerometer report to a microprocessing chip, the chip will distinguish the singles from the entry point of the information to the chip.

MEMS Accelerometer Formula

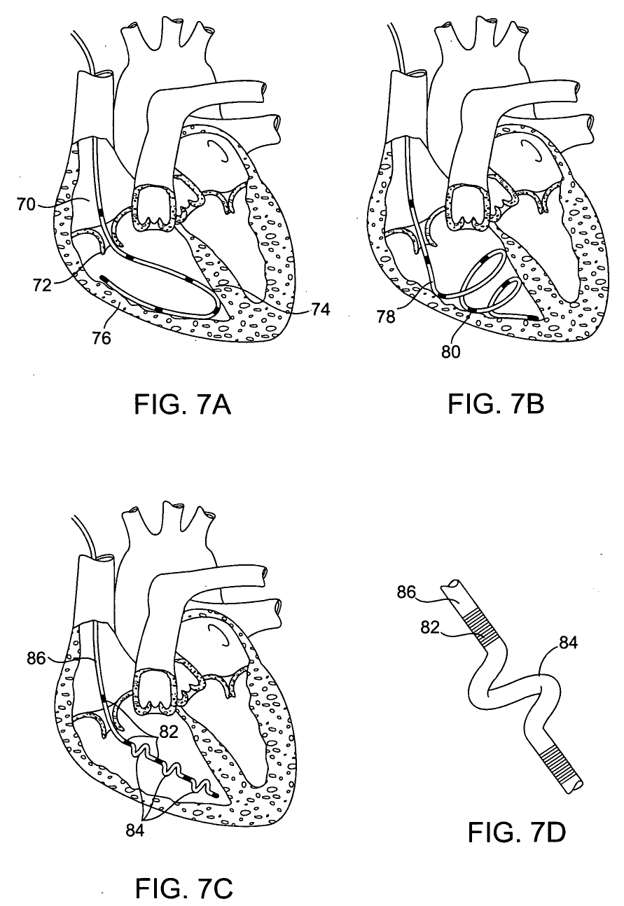

Elwenspoek et. al. at MESA Research Institute have provided description of generalized designs for certain MEMS accelerometers as well as the basic physics principles underlying them (Mechanical Microsensors, Springer-Verlag, Berlin, 2000, pp 133-143). Silicon accelerometers generally consist of a proof mass M which is attached to a fixed frame by one or more spring elements. FIG. 7.1 shows a simple lumped element model of such a structure.

[image]

In this model, K is the effective spring constant of the spring element(s) and D is the damping factor. The operation of the device is based on Newton's second law of motion:

$$F = \frac{ⅆ p}{ⅆ t} = Ma \qquad ( 7.1 )$$

where F is the force acting on the mass p is the impulse momentum and a is the acceleration of the mass. From (7.1) it is clear that an acceleration results in a force being exerted on the mass. This force results in a deformation of the spring element(s) and a displacement of the mass given by:

$${d}_{static} = \frac{F}{K} = \frac{Ma}{K} \qquad ( 7.2 )$$

The subscript static indicates that (7.2) is only valid for slow variations of the acceleration, i.e. well below the resonance frequency of the system.

The dynamic behavior of the system can be analyzed by considering the differential equation:

$$M \frac{{ⅆ}^{2} x}{ⅆ {t}^{2}} + D \frac{ⅆ x}{ⅆ t} + Kx = {F}_{ext} = Ma \qquad ( 7.3 )$$

where Fₑₓₜ, is the external force acting on the reference frame to which the proof mass is attached. Using Laplace transformation, the following second-order mechanical transfer function from an acceleration to a displacement of the mass is obtained:

$$H ( s ) = \frac{X ( s )}{A ( s )} = \frac{1}{{s}^{2} + \frac{D}{M} s + \frac{K}{M}} = \frac{1}{{s}^{2} + \frac{{ω}_{r}}{Q} s + {ω}_{r}^{2}} \qquad ( 7.4 )$$

Where

$${ω}_{r} = \sqrt{\frac{K}{M}}$$

is the resonance frequency and

$$Q = \frac{{ω}_{r} M}{D}$$

is the quality factor. From this equation we see that we can also write (7.2) in terms of the resonance frequency:

$${d}_{static} = \frac{a}{{ω}_{r}^{2}} \qquad ( 7.5 )$$

which clearly illustrates the trade-off between bandwidth and sensitivity. For a high dc sensitivity we need a low resonance frequency. Feedback may be used to eliminate this trade-off as will be explained later. The performance of accelerometers is limited by the thermal motion of the proof mass. According to the laws of thermodynamics, the thermal energy of a system-in equilibrium is k_{B}T/2 for each energy storage mode, where k_{B} is the Boltzmann constant and T is the temperature. The small proof mass of micromachined accelerometers results in rather large movements. An equivalent acceleration spectral density, the so-called total noise equivalent acceleration (TNEA) can be calculated and is given by (Gabrielson 1993):

$$TNEA = \sqrt{\frac{\overline{{a}_{n}^{2}}}{Δ f}} = \frac{\sqrt{4 {k}_{B} TD}}{M} = \sqrt{\frac{4 {k}_{B} T {ω}_{r}}{QM}} \qquad ( 7.6 )$$

It is clear that for measuring low acceleration levels a large proof mass and high quality factor are required.
Fabrication

In the case of capacitive devices there is a trade-off between large capacitance values and low damping: the narrow gap required for a large capacitance also results in large damping. Therefore, bulk micromachined devices typically require packaging at a prescribed pressure to control the damping. Surface micromachining allows integration in a single wafer and it can be easily combined with integrated electronic interface circuits. Furthermore, the thin structures allow for the realization of arrays of holes to control the damping.

Just like bulk micromachined devices, surface micromachined accelerometers can be distinguished by their sensitive axis. In a so-called vertical structure the mass consists of a plate which moves in a direction perpendicular to the wafer surface and the mass forms a parallel plate capacitor with an electrode beneath it. In lateral substructures, i.e. with the sensitive axis parallel to the wafer surface, a comb structure is attached to the mass and movements of the mass are sensed by measuring the capacitance between the fingers attached to the mass and fixed fingers attached to the substrate.

The vertical structure has the advantage of a significantly larger capacitance value: up to 1 pF compared to less than 200 fF for lateral structures. However, the structure is asymmetric and a voltage between the capacitor plates will result in an electrostatic force pulling the mass towards the wafer surface.

[image]

[image]

Because of the low inertial mass, very soft springs are needed to achieve a detectable displacement of the sensor. However, extremely weak springs are impractical because of sagging due to gravitation, susceptibility to self-resonance, and a tendency to stick to the substrate. Therefore, the folded beam suspension used by Lu et al. was dimensioned to obtain a vertical spring constant of 1.1 N/m. Thus, a 10 mg acceleration results in a displacement of only 0.05 nm and an extremely sensitive position sensing circuit is required.

As mentioned before, a problem with vertical structures is that the voltage used to detect capacitance changes also exerts an electrostatic pull-down force. Lu et. al. solved this problem with the help of a comb structure around the periphery of the proof-mass. The comb fingers generate an opposing force to maintain the nominal position of the sensing element (Tang 1992). This is illustrated in FIG. 7.8. The silicon surface acts as a ground plane, resulting in an asymmetric electrical field distribution around the moveable finger. The result is a net pull-up force, which for small displacements is approximately proportional to the square of the applied bias voltage. An alternative solution is to use an additional electrode above the proof-mass, however this requires a more complex fabrication process.

[image]

FIG. 7.9 shows the basic structure of a lateral accelerometer, i.e. with the sensitive axis in the wafer plane. This is also the structure used in the well-known accelerometers available from Analog Devices (Analog Devices 1995). The structure consists of a proof mass which is suspended to two anchors with U-shaped springs. Again, the mass contains a large number of holes to ensure complete etching of the sacrificial oxide layer underneath it. Contrary to the z-axis structure, the holes do not influence the damping as the mass now moves in the wafer plane. Comb structures are attached to the mass to both sense the position and exert electrostatic forces. The latter can be used for force feedback (see Sect. 7.1.4) and self test functions.

[image]

Of course, the realization of one vertical and two lateral accelerometers allows for the realization of a three axis accelerometer on a single chip. Such a chip was realized by Lemkin et al. (Lemkin 1997, 1999). They combined the surface micromachined accelerometer structures with a 2 μm CMOS process to realize the interface electronics. FIG. 7.10 shows a photograph of the complete accelerometer chip where the accelerometer structures are clearly visible in the center of the chip.

As mentioned before, surface micromachined accelerometers have several advantages compared to bulk micromachined devices. However, one major disadvantage is the significantly smaller proof mass, which results in a much higher noise level. Because of their large proof mass, bulk-micromachined accelerometers obtain a high sensitivity with an equivalent noise level (eqn. (7.6)) below 1 μg/\sqrt{Hz} over a bandwidth from dc up to 100 Hz.

For a typical surface micromachined accelerometer the noise level is more than a hundred times larger. For many commercial applications (e.g. in the automotive sector) this is still acceptable, but inertial navigation and other precision applications such as tracking systems for head-mounted displays demand better performance (Boser 1996). A solution to this problem was proposed by Yazdi et al. (Yazdi 1999). They used a combination of bulk and surface micromachining to realize the accelerometer structure indicated in FIG. 7.11. Two polysilicon structural layers are used to realize a fixed electrode between two moving electrodes.

The proof mass is suspended by a number of beams in the top polysilicon layer. The bottom poly layer forms an electrode between the proof mass and the top layer. This middle electrode is made rigid by embedding thick vertical stiffeners in it. These stiffeners are formed by thin film deposition and refilling of high aspect-ratio trenches in the proof mass (Selvakumar 1994). The top electrode is made rigid by making it short and wide, and supporting it through electrically-isolated standoff on the proof mass. These standoffs are formed by the first poly layer and the dielectric layers on top and bottom of it. The sacrificial oxide layers between the proof mass and first poly and between the two electrodes are sealed by the poly layers and kept at the anchors to bring the anchor height to the level of the second poly.

[image]

[image]

One method appropriate for fabrication of micromachined accelerometers to produced a Lateral CMOS-MEMS Accelerometer has been described by Gary Fedder's group at CMU (IEEE MEMS 2000, Jan. 23-27 2000 Miyazaki Japan).

The photo below shows the second generation CMOS-MEMS accelerometer fabricated at CMU. The size of device is about 320 μm by 420 μm and it weighs only about 0.4 μg! This lateral accelerometer consists of proof-mass (middle) and serpentine springs at the two ends of the proof-mass. The latticed structures are the release holes to make sure the silicon underneath can be etched away. The whole device is anchored on the substrate. The electronics are integrated on the same substrate (not shown in this picture).

[image]

Another method appropriate for fabrication of micromachined accelerometers has been described by Huikai et. al. in “Design and Fabrication of An Integrated CMOS-MEMS 3-Axis Accelerometer” Nanotech 2003. Vol. 2

The process flow is shown in FIG. 1. The CMOS chip is etched from its back side, leaving a 10 pm to 100 pm-thick single-crystalline silicon (SCS) membrane (Fig. I(a)). This backside deep Si etch step is used to control the thickness of microstructures. Next, an anisotropic dielectric etch is performed from the front side using the top metal layers as etching mask (Fig. I(b)). Then, another deep Si etch is used to release microstructures (Fig. I(c)). The last isotropic Si etch step (Fig. I(d)) provides a specific undercut of bulk Si to create compliant structures along with thick, stiff Si structures

[image]

3D Accelerometer Fabrication

In certain embodiments of the present invention, it is useful to employ accelerometers with sensing capability. By example, the relative vertical motion of accelerometers which have been positioned within the same cardiac wall gives information regarding the shortening of that cardiac feature. Through analysis of this information, one can extrapolate the changes this shortening can effect on the heart chamber dimensions and multi-dimensional volume.

Accelerometers with three-axis motion sensing capability are also useful when modeling or remodeling of heart features change the relative position of the inventive accelerometers. These physical changes to the heart can occur as a result of successful therapy or progressive disease. Three-axis motion sensing accelerometers in such cases are useful in assessing the new relative positions of the accelerometers, and making appropriate accommodations for these changes. When physically associated with other devices, such as pacing electrodes, they can also give information on the new relative location of these physically proximate devices. In some cases, three orthogonal chips to obtain 3-D resolution are not required.

An important advantage of employing a 3-axis accelerometer in the inventive wall position detector system is that the physician need not orient the implant in any specific orientation relative to the heart wall during surgery. With a 3-axis accelerometer, the vector sum of the accelerations in each axis can be calculated to determine the magnitude of the heart wall acceleration.

The principle of lateral-axis and z-axis capacitive comb finger sensing is illustrated in FIG. 2. If the metal layers on the stators and rotors are electrically connected, respectively, the CMOS comb drive functions just like a lateral-axis polysilicon comb drive (FIG. 2(a)). If all three metal layers in the stators are electrically connected while the metal-1 and metal-3 in the rotors are separately connected, two sidewall capacitors, C, and C2, are formed, as shown in FIG. 2(b). When the rotor moves due to an external acceleration, C, and C2 will change values in opposite directions. Because of the high wiring flexibility, a fully differential capacitive bridge can be easily formed. Such a z-axis accelerometer in a thin-film CMOS-MEMS process was previously demonstrated. All the comb fingers have a “T” shape cross-section due to the silicon undercut. As shown in FIG. 3, electrodes are only placed at the sidewall edges to reduce the parasitic capacitance overlap to the silicon layer. The parasitics can be further reduced by using narrower silicon layer. The width of the silicon layer is controlled by the silicon undercut (see Fig. I(d)). The silicon layer should not be too narrow (−2 m) as the mechanical robustness over manufacturing variations must be maintained.

[image]

[image]

Accelerometer Lead System to Measure Local Timing and Magnitude of Cardiac Wall Contractions

The present inventive implantable wall position detector system makes use of an accelerometer lead system in order to measure timing and magnitude of local cardiac wall contractions. This contractile timing information may be of great use in the optimization of cardiac resynchronization therapy for congestive heart failure patients.

As discussed in another Proteus Biomedical patent reference above, the placement of accelerometers on the left ventricle cardiac vein, septum, and the right ventricle free wall can give valuable information about the timing of the contractions between these distinct heart walls.

The present inventive implantable wall position detector system describes accelerometers that are used to measure local contractions in specific regions of the heart. In addition to optimization of CRT, for congestive heart failure patients, this invention could be used for monitoring of the development of local ischemia.

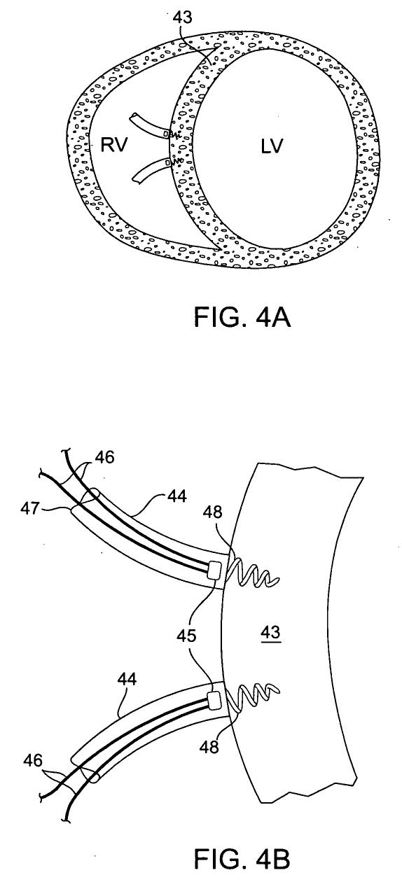

FIG. 4A showing one embodiment of the inventive implantable wall position detector shows a transverse section of the heart 40 where various components of the heart are visible such as the right ventricle the left ventricle, and the interventricular septum 43. Also shown is a pair of accelerometer leads which have been placed in the mid interventricular septal wall. The leads are placed preferably within about 1.0 to 3.0 cm apart in order to measure the local timing and magnitude of contractions in that specific region of the heart wall.

FIG. 4B shows in greater detail the accelerometer leads 44 in one embodiment of the inventive implantable wall position detector, that have been place in the interventricular septal wall 43. The accelerometer sensors which could be a 1 axis, 2 axis, or 3 axis accelerometers 45 are positioned at the distal tip of the accelerometer leads 44 and are connected to several conductor wires 46 which connect to the proximal end of the accelerometer leads 44.

These components are then housed within the lead body 47 which is preferably made of a standard lead material such as polyurethane or silicone. At the most distal tip of the lead is an active fixation helix 48 which is used to anchor the lead into the cardiac wall tissue.

The accelerometer leads 44 are implanted under fluoroscopy using standard permanent pacing lead delivery techniques which include introducers, guide catheters, guide wires, and stylets.

Once the accelerometer leads 44 have been guided to the desired region for measurement, they are twisted into place, such that the active fixation helix 48 engages the cardiac wall tissue thereby securing the lead to the cardiac tissue.

A second accelerometer lead is placed within proximity of the first lead such that the area spanning the two accelerometers can be measured for timing and magnitude of contractions. Any dyssynchronous contractions between the two accelerometers leads is detected by comparing the accelerometers signals.

Contractile synchrony of the two accelerometer leads is visible as similar timing of accelerometer signals measured from each lead. A dyssynchronous contraction between the two leads would be visible as a delay between the timing of accelerometer signals. Furthermore, the timing information from these accelerometers leads could be compared to any other timing devices placed within the heart.

In one embodiment of the implantable wall position detector, an initial measurement using fluoroscopy is made of the distance between the two distal tips of the lead at the time of implantation. By knowing the initial position and orientation of the accelerometers and then double integrating the accelerometer signals, it is possible to calculate position and therefore changes in distance between the two accelerometers, and thereby determine strain of contractions.

In addition to measuring the local strain of the contractions between the two accelerometers, the clinician will be able to infer changes in cardiac wall thickness using the implantable wall position detector. Assuming that the cardiac tissue is incompressible, when the tissue contracts thickening and shortening are directly correlated. For example, as the tissue contracts and shortens, in order for it to maintain a constant volume it has to thicken. Therefore a device such as described in this present invention, that attaches to the surface of the tissue can be used to measure surface strain and also calculate changes in tissue thickness.

One important advantage of the embodiment of the inventive implantable wall position detector device provided in this figure is that it does not require invasive placement deep into the tissue. Further, it does not require a device on both sides of the tissue wall. This information is especially useful not just for optimization of CRT but also in monitoring the appearance of tissue ischemia.

Knowledge of the orientation of the accelerometer leads can be useful in being able to precisely calculate the changes in distance between the two accelerometers which is used to derive the local strain measurements. One method to know the orientation and position of the leads is to place a number of radio opaque markers at the distal end of the lead and use of fluoroscopy. Furthermore the tilt information provided by the accelerometer because of its sensitivity to gravity provides additional orientation information.

In addition to providing local timing and magnitude of cardiac wall contractions, this inventive embodiment of the implantable wall position detector can provide more global information about the motion of the cardiac wall. Indeed the accelerometer in each accelerometer lead will sense any overall motion of the cardiac wall.

Another advantage in this implantable wall position detector design embodiment is that the two points that are being used to measure timing and magnitude of contractions are independent and free to move relative to each other and thereby they do not constrain any motion of the cardiac wall.

A number of other local strain devices have been disclosed in the prior art. However, these prior art devices generally include two points of measurement which are directly attached to each other thereby constraining the wall from contracting in a natural manner.

As mentioned above, the present invention has multiple clinical applications. These include optimization of CFT in congestive heart failure patients as well as monitoring the development local ischemia, among other. In the case of CRT optimization, the local timing of the contractions provided by the accelerometer leads can be compared with timing of other timing devices such as strain gauge leads placed within the left ventricle cardiac vein or right ventricle free wall.

For the clinical application of monitoring local ischemia, the information provided by the accelerometer leads concerning local strain and magnitude of contractions would be very valuable. A decrease in the calculated contractile strain or decrease in the contractile thickening of the heart wall as measured by the accelerometer leads would be indicative of the development of ischemia in that region of the accelerometer leads.

Wireless and Battery Free Accelerometer Sensor

An additional embodiment of the present inventive implantable wall position detector is a wireless externally powered cardiac implant to measure acceleration. These wireless devices can be implanted throughout the heart and have unique value in cardiac resynchronization therapy.

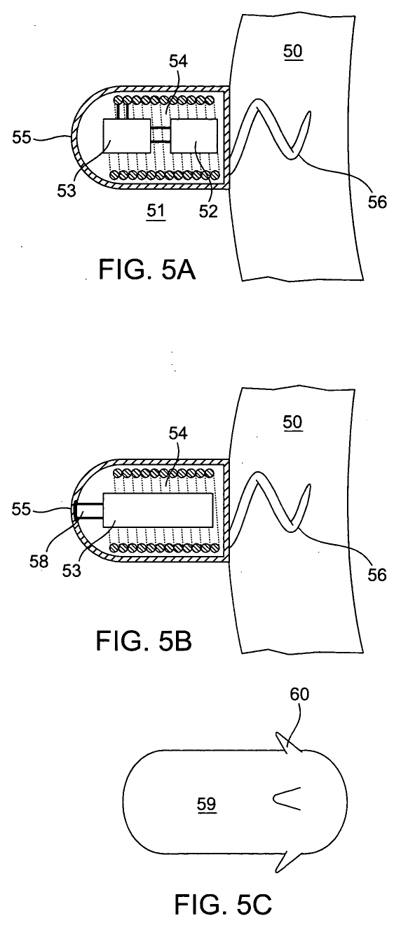

FIG. 5A shows an embodiment of the present inventive implantable wall position detector which is a wireless and battery free accelerometer sensor 51. It is comprised of several components which include an accelerometer sensor 52, which is connected to electronics 53. Electronics 53 are also connected to a coil 54. All of these components are enclosed within a hermetic housing 55. On the distal end of the device is an active fixation helix 56 which is used to anchor the device into the cardiac wall tissue 50.

The accelerometer 52 of the present inventive implantable wall position detector can be a single, dual or triple axis accelerometer. The coil 54 is used for both power and data transmission via magnetic inductive coupling between the coil 54 and an external coil not shown in this figure. This external coil would be included in an interrogator type device.

The coil 54 is connected to the electronics which includes a micro-processor and RF DC converter. This takes the RF energy being transmitted by the external coil and the external interrogator and converts it into DC power for both the microprocessor and the sensor. The electronics 53 also includes a modulator, and a demodulator for the RF telemetry or data transmission and receiving of data.

The hermetic enclosure 55 can be made of a biocompatible material such as titanium and laser welded to form a hermetic enclosure. On the distal end, the active fixation helix 56 can also be formed from a biocompatible metal such as titanium and then welded to the hermetic enclosure 55.

This embodiment of the inventive present inventive implantable wall position detector device can be installed in the heart using standard permanent pacing lead delivery systems for the heart. These include introducers, guide catheters, guide wires, and stylets, among others.

A custom delivery catheter (not shown) can be used in parallel with the other delivery tools. This delivery catheter is able to hold the wireless sensor at its distal tip during placement. Within the delivery catheter is a stylet type device. This device which can engage the proximal end of the wireless sensor 51, thereby screwing it into place into the cardiac wall 50. The delivery catheter then disengaging from the wireless sensor 51, leaving it behind anchored to the cardiac wall with the active fixation helix 56.

As described above, in a physician's office an interrogator would be placed on the chest of the patient and used to both power and transmit data from the wireless sensor. The physician can then use information from this accelerometer sensor as well as the other sensors used in concert with the accelerometer in order to optimize cardiac resynchronization therapy.

FIG. 5b shows a wireless pressure sensor 57. Wireless pressure sensor 57 has many of the components of the wireless accelerometer sensor, with the exception that the accelerometer 52 has been replaced with a pressure sensor 58. Furthermore, instead of being completely enclosed within the hermetic enclosure 55, the pressure sensor sits on or just under the surface of the hermetic enclosure 55 in order to measure surrounding pressure.

Both the wireless accelerometer sensor and wireless pressure sensor as described above can be placed in numerous areas within the heart where they can be screwed and anchored into place. However, these designs may not be as suitable for the cardiac vein on the epicardial surface of the heart.

FIG. 5c shows a device 59 which has been designed to be placed in the cardiac vein of the left ventricle. It contains a sensor such as an accelerometer, electronics, coil for both power and data transmission and an hermetically sealed enclosure. The outside surface of the device is smooth. In order to be atrematic only soft silicone tines 60 are protruding from the device. Soft silicone tines 60 stabilize device 59 once it has been placed in the cardiac vein.

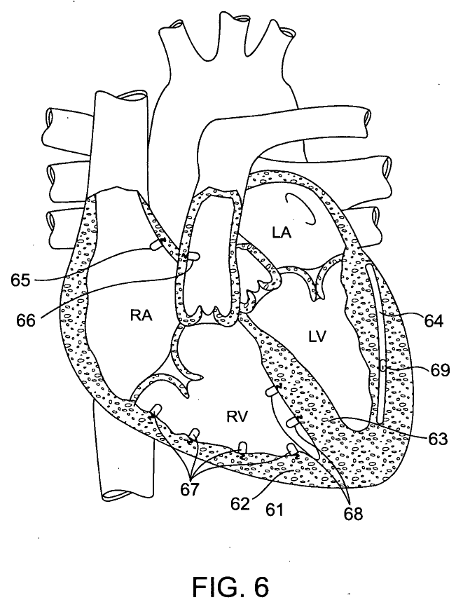

FIG. 6 shows a section view of the heart 61. Visible are the right atrium, right ventricle, left atrium, left ventricle and coronary artery as well as the right ventricle free wall 62, the interventricular septum 63, and the cardiac vein of the left ventricle 64.

Wireless sensors with active fixation helixes 65, 66, 67, and 68 are shown anchored in the right atrium, pulmonary artery, right ventricle free wall, and right ventricle interventricular septal wall respectively. A wireless sensor capsule with tines 69 is shown place within the cardiac vein 64 of the left ventricle. All of these placements work synergistically to give a very comprehensive picture of the heart contractile timing.

One of the advantages of the battery free design is that there are no battery hazards or battery life issue connected with this device. The main advantage of the device comes from its battery free nature and small size. This allows placement of a multitude of these devices throughout the heart without impeding future placement of other cardiac devices. Such further cardiac devices include permanent pacing leads in the right ventricle, right atrium, and cardiac vein of the left ventricle, among others.

FIGS. 7A-D provide views of the inventive device in a shaped set lead with multiple displacement sensors and/or electrodes. In this signal lead, motion between various structures of the heart (i.e. lateral ventricle wall and interventricular septum), can be measured by continuously measuring distance between the electrodes using independent voltage amplitudes, time of light, or different sensors. Shaped set lead 70 extends though the right atrial valve 72, proceeding to the right ventricle, where shaped set lead curves between the interventricular septum wall 74 and the ventricular free wall 76. In this position, the shaped lead 70 will flex with the cardiac contractions, but remained positioned relative to the walls in order to provide accurate information as to their position.

In FIG. 7B is shown an additional approach to the embodiment above. In this approach, the shaped set lead 78 is provided in a spiral configuration, providing a three dimensional positioning form to support and position electrodes or sensors 80.

In FIG. 7C is shown electrodes or sensors 82 along a lead 86 which is provided with segmented spiral shaped flexible coils 84.

FIG. 7D provides an enlarged view of the isolating segmented spiral flexible coils 84 sections of lead 86. The motion of the electrode/sensors 82 will be more isolated and independent with these spirals, and will follow the direction of the lead proper.

These inventive configuration, when used singly or in concert, can optimize freedom between the electrodes and sensors in the desired directions.

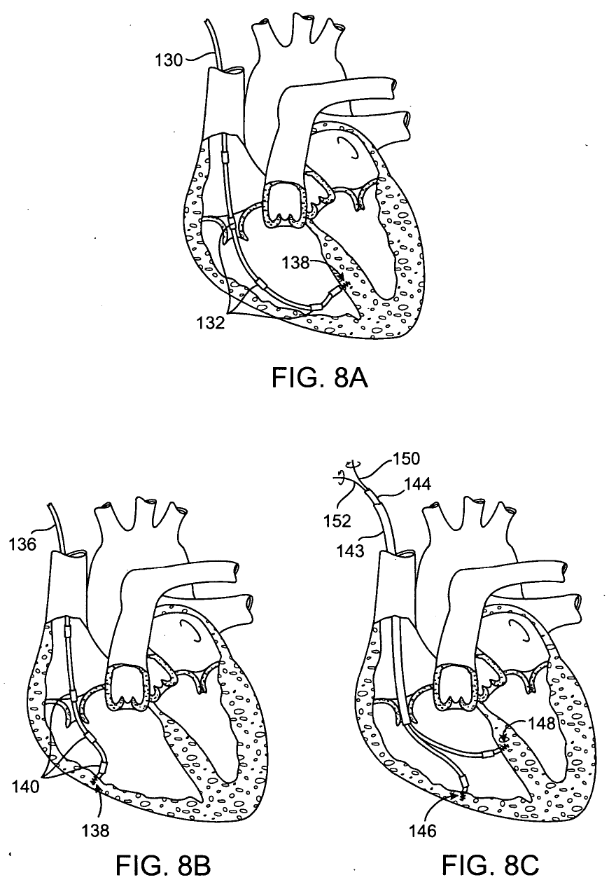

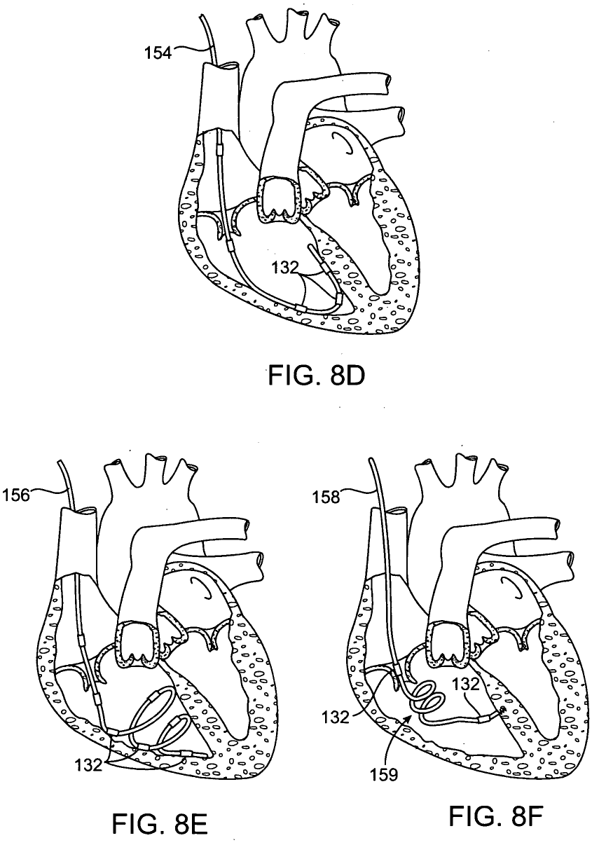

FIG. 8A provides a cross-sectional view of an accelerometer lead embodiment of the inventive cardiac timing device. Accelerometer lead 130 is placed in the right ventricle of the heart with an active fixation helix at the end which is embedded into the cardiac septum. In this view, the accelerometer lead 130 is provided with one or multiple accelerometers 132. The distal tip of the accelerometer lead 130 is provided with an active fixation helix 138 which is screwed into the mid-septum 134.

Accelerometer lead 130 is placed in the heart in a procedure similar to the typical placement procedures for cardiac right ventricle leads. Accelerometer lead 130 is placed in the heart using the standard cardiac lead devices which include introducers, guide catheters, guidewires, and/or stylets. Accelerometer lead 130 is inserted into the clavicle vein, thru the superior vena cava, through the right atrium and down into the right ventricle. Accelerometer lead 130 is positioned under fluoroscopy into the location the clinician has determined is clinically optimal and logistically practical for fixating the accelerometer lead 130 and obtaining motion timing information for the cardiac feature area surrounding the attachment site. Under fluoroscopy, the active fixation helix 138 is advanced and screwed into the cardiac tissue to secure accelerometer lead 130 onto the septum.

Once the accelerometer lead 130 is fixed on the septum, accelerometer lead 130 provides timing data for the regional contractile motion of the septum. The accelerometers 132 which are located more proximally along accelerometer lead 130 provide timing data on the regional accelerations in those areas of the heart. By example, an accelerometer 132 situated near the AV valve, which spans the right atrium in the right ventricle, provides timing data regarding the closing and opening of the valve. Furthermore, accelerometers 132 situated along other portions of the lead could provide local blood flow data by measuring the vibrations of the lead induced by blood flow. Such information is important in provided cardiac timing determinations.

In a preferred embodiment, accelerometer lead 130 is constructed with the standard materials for a cardiac lead such as silicone or polyurethane for the lead body, and MP35N for the coiled or stranded conductors connected to the accelerometers 132. Alternatively, these device components can be connected by a multiplex system, as describe above, to the proximal end of accelerometer lead 130. The proximal end of accelerometer lead 130 connects to a data acquisition device. The data acquisition device is comprised of several devices such as an amplifier, a A/D converter, a microprocessor and other display units to provide timing data from the various accelerometers 132.

The accelerometer lead 130 is typically fabricated of a soft flexible lead with the capacity to conform to the shape of the heart chamber. The only fixation point in this embodiment of the present cardiac timing device is the active fixation helix 138 which is attaching the accelerometer lead 130 to the cardiac septum.

FIG. 18B provides a view of an accelerometer lead 136 with an active fixation helix 138 on its distal end, but with a different site of attachment. Accelerometer lead 136 has one or more accelerometers 140 embedded along its length. Accelerometer lead 136 is physically identical to accelerometer lead 130 shown in FIG. 18A. The primary difference between these two views is that in this the distal end of the accelerometer lead is screwed into the lateral wall of the right ventricle.

The clinical motivation for these fixation alternatives is to provide cardiac timing information via accelerometer leads 136 and 130 about the contractile motions of the cardiac tissue where they are fixated. In FIG. 18A, the accelerometer lead 130 attached to the septum will provide cardiac timing data primarily for septal motion. In FIG. 18B accelerometer lead 136 is attached to the lateral wall of the right ventricle, and will give cardiac timing data primarily regarding the motion of that portion of the heart.

FIG. 18c provides a view of a bifurcated accelerometer lead 144 being placed with a guide catheter 143. In order to place the bifurcated accelerometer lead 144, the guide catheter 143 tip is first placed into the right ventricle and the bifurcated accelerometer lead 144 is slowly advanced through the guide catheter 143. As the bifurcated accelerometer lead 144 enters into the right ventricle, it is released from the laterally confining guide catheter 143, and unfurls into its intrinsic bifurcated shape. Under fluoroscopy, bifurcated accelerometer lead 144 is advanced until the two distal tips 146 and 148 are in the desired location on the heart, such as right lateral wall location 146 and septal wall location 148. Once distal tips 146 and 148 are in a desired position, torque wires 150 and 152 are used to advance the active fixation helixes and screw them into the tissue. Alternatively, passive fixation with tines can be employed to stabilize bifurcated accelerometer lead 144.

The inventive embodiment described in FIG. 18C enjoys number of advantages over the non-bifurcated embodiments. The bifurcated configuration of the inventive cardiac timing device allows, in a single deployment procedure, the placement of two active fixation helixes on two different regions of the heart. Thus, a considerable increase in cardiac timing information can be obtained in a single procedure. An additional advantage of this device configuration is that there is a more controlled reference position between distal tips 146 and 148 than would be available with individual placement. Also, for accelerometers which are measuring motion within the ventricle, the bifurcated type placement allows more predictable and stable positioning.

FIG. 18B provides a view of a U-shaped accelerometer lead 154. This diagram shows the position of U-shaped accelerometer lead 154 after deployment in the right ventricle. U-shaped accelerometer lead 154 is provided with one or more accelerometers 132 along its length. The main motivation for U-shape configuration is to guarantee contact of the accelerometer lead with two walls of the heart, such as the septal wall and the right ventricle lateral wall.

U-shape accelerometer lead 154 is deployed using a guide catheter which would be placed into the right ventricle. The straightened U-shape accelerometer lead 154 is then slowly advanced out of the guide catheter. As it exits the guide catheter, U-shape accelerometer lead 154 assumes its intrinsic U-shape within the right ventricle. Alternatively, a straight stylet placed within U-shape accelerometer lead 154 can be used to hold the lead in a straight position during initial right ventricle placement. Once the lead is placed in the right ventricle, the stylet is removed and U-shape accelerometer lead 154 assumes its intrinsic U-shape.

The fabrication of the U-shape can be accomplished through a number of known methods. By example, the silicone lead body can be molded as a U-shape during the processing. Alternatively, the metal conductor coils or strands within the lead body can be shape set into a U-shape using various heat treatment methods.

U-shaped accelerometer lead 154 may optionally include an active fixation helix (not shown) along the length of the lead to fixate it as shown in the figures above. However, such additional fixation need only be provided when there is an unusually demanding cardiac feature target area preferred for fixation, or the point of attachment needs to be highly precise. The most preferred embodiment of U-shaped accelerometer lead 154 does not require an active fixation, but by the nature of its U-shape will hold this position within the ventricle chamber of the heart.

It is a theory of the inventors that, during systole and diastole of the heart, the U-shaped accelerometer lead 154 flexes back and fourth and shifts slowly up and down.

FIG. 18E provides a view of spiral accelerometer lead 156. As with the examples above, spiral accelerometer lead 156 includes one or more accelerometers 132 embedded along its length. Spiral lead 56 would be deployed using similar guide catheter and stylet methods as described for the U-shape accelerometer lead 154. As with U-shaped accelerometer lead 154, the primary purpose of the spiral shaped lead is to guarantee contact with the side walls of right ventricle chamber. In this case when the chamber contracts, the spiral lead would flex and motion would be measured on its one or more accelerometers. The accelerometers provide regional timing and displacement information at the various positions where the accelerometers come in contact with the right ventricle walls. Another option is to have an active fixation helix on the distal tip, but in the preferred embodiment shown in FIG. 18E, there is no active fixation.

FIG. 18f shows accelerometer lead 158 with coil segment 159. In this view, the accelerometer lead 158 has been installed within the right ventricle and right atrium. The accelerometers 132 are separated from one another by coil segment 159 on the accelerometer lead 158. The function of coil segment 159 is to create independent or free floating segments of the lead which can flex without influencing each other. By example, in some of the previous configurations, there is the potential for direct influence of movement of one portion of lead on another simply due to the natural stiffness of the lead. The main goal of the embodiment shown in FIG. 18F is to isolate these segments using a very flexible coiled segment 159 in the accelerometer lead 158. One of the advantages of a multiple coil device configuration is that multiple coiled segments provide substantial mechanical isolation of the different accelerometers 132 from each other. The cardiac timing data then received from the various accelerometers 132 then give highly accurate data specific for regional motions in the heart along the accelerometer lead 158.

What is claimed is:

1. A method for evaluating movement of a cardiac tissue location in a subject, said method comprising:
(a) determining timing of a signal obtained from a accelerometer stably associated with said cardiac location; and
(b) using said determined timing to evaluate movement of said cardiac tissue location.

2. The method according to claim 1, wherein said method comprises using timing of signals produced by two or more accelerometers each stably associated with a different cardiac tissue location.

3. The method according to claim 2, wherein said accelerometer is present on a lead.

4. The method according to claim 3, wherein said lead comprises two or more accelerometers positioned at different locations at a distal end of said lead.

5. The method according to claim 1, wherein said accelerometer comprises an apical accelerometer.

6. The method according to claim 1, wherein said accelerometer comprises a one axis or multi axis accelerometer

7. The method according to claim 1, wherein said accelerometer comprises at least one existing electrodes or other cardiac elements which can serve in an accelerometer capacity.

8. The method according to claim 7, wherein said electrode was not initially designed or implanted to serve in the capacity of an accelerometer.

9. The method according to claim 1, wherein said accelerometer provides full time analysis via download, or real time interrogation on an external basis.

10. The method according to claim 1, wherein said accelerometer comprises a transducer that is hermetically sealed.

11. The method according to claim 1, wherein said accelerometer comprises a transducer that is not hermetically sealed.

12. The method according to claim 7, wherein said transducer is fabricated from a piezoelectric material.

13. The method according to claim 3 wherein the timing and displacement of contraction of the monitored sections of the heart are compared to one another, phase and amplitude differences evaluated, and means manually or automatically taken to move contraction of wall segments into synchronization with one another.

14. The method according to claim 3, wherein said accelerometer is coupled to a signal conductive element present in said lead.

15. The method according to claim 14, wherein said signal conductive element is an electrically conductive element.

16. The method according to claim 3, wherein said accelerometer is about 0.25 mm in width, preferably about 0.25 to 1.5 mm wide and most preferably about 0.25 to 1.0 mm wide.

17. The method according to claim 3, wherein said lead comprises a tissue securing element for stably associating said accelerometer with said cardiac tissue location.

18. The method according to claim 3, wherein said lead is configured so that said accelerometer is stably associated with said cardiac location by a compressive force.

19. The method according to claim 1, wherein said accelerometer is present on an acute device.

20. The method according to claim 1, wherein said accelerometer is present on a permanently implantable device.

21. The method according to claim 1, wherein said method comprises further obtaining a second motion timing signal from a second cardiac location.

22. The method according to claim 21, wherein said second motion timing signal is obtained using a magnetic induction sensing element.

23. The method according to claim 22, wherein said second cardiac location is a septal wall location.

24. The method according to claim 23, wherein said cardiac location is a heart wall location.

25. The method according to claim 24, wherein said heart wall is a chamber wall.

26. The method according to claim 25, wherein said chamber wall is a ventricular wall.

27. The method according to claim 25, wherein said chamber wall is a septal wall.

28. The method according to claim 1, wherein said method is a method of determining timing of cardiac wall motion.

29. The method according to claim 28, wherein said method is a method of determining cardiac wall motion of a first cardiac wall relative to a second cardiac wall.

30. The method according to claim 29, wherein said method is a method of determining timing of cardiac wall motion of a first cardiac wall relative to a second cardiac wall.

31. The method according to claim 30, wherein said method is a method of detecting ventricular mechanical dyssynchrony.

32. The method according to claim 31, wherein said ventricular mechanical dyssynchrony is interventricular.

33. The method according to claim 31, wherein said ventricular mechanical dyssynchrony is intraventricular.

34. The method according to claim 31, wherein said method further comprises performing cardiac resynchronization therapy based on said detected dyssynchrony.

35. A system for evaluating movement of a cardiac tissue location, said system comprising:
(a) a accelerometer stably associated with said cardiac tissue location; and
(b) a signal processing element configured to employ timing of a signal obtained from said accelerometer that is induced by movement of said cardiac tissue location to evaluate movement of said tissue location.

36. A computer readable storage medium having a processing program stored thereon, wherein said processing program operates a processor to operate a system according to claim 38 to perform a method according to claim 1.

37. A processor comprising a computer readable medium according to claim 36.

38. A kit comprising:
a computer readable storage medium according to claim 36.

39. The kit according to claim 38, wherein said computer readable storage medium is present in a processor according to claim 37.

40. The kit according to claim 38, wherein said processor is present in a cardiac pacing device.

41. A device for evaluating movement of a cardiac location, said device comprising:
(a) an accelerometer stably associated with said cardiac tissue location; and
(b) a signal processing element configured to employ timing of a signal obtained from said accelerometer that is induced by movement of said cardiac tissue location to evaluate movement of said tissue location.

42. The device according to claim 41, wherein said device further comprises a cardiac electrical stimulation element.

43. The device according to claim 42, wherein said device is a cardiac resynchronization therapy device.

44. The device according to claim 41 wherein said accelerometers are provided in trapezoid shaped sensor to allow visualization of specific orientation for an axis of the sensor.

45. The device according to claim 44 wherein said accelerometers are single axis with long dimension of about 0.25 to 10.0 mm, preferably about 0.5 to 5.0 mm, and most preferably about 1.0 mm to 5.0 mm.
